SEQUENCE LISTING (18 sequences)

SEQ ID NO: 1 - protein, 24 aa, Homo sapiens
MAPRGFSCLLLLTSEIDLPVKRRA

SEQ ID NO: 2 - protein, 24 aa, Homo sapiens
MAPRGFSCLLLLTGEIDLPVKRRA

SEQ ID NO: 3 - protein, 24 aa, Homo sapiens
MAPRGFSALLLLTSEIDLPVKRRA

SEQ ID NO: 4 - protein, 24 aa, Homo sapiens
  MISC_FEATURE (14)..(14): D-serine
MAPAGASCLLLLTSEIDLPVKRRA

SEQ ID NO: 5 - protein, 24 aa, Homo sapiens
MAPAGASCLLLLTGEIDLPVKRRA

SEQ ID NO: 6 - protein, 24 aa, Homo sapiens
  MISC_FEATURE (14)..(14): D-serine
MAPAGASCLLLLTSEIDLPVKRRA

SEQ ID NO: 7 - protein, 17 aa, Homo sapiens
  MISC_FEATURE (12)..(12): D-serine
PAGASCLLLLTSEIDLP

SEQ ID NO: 8 - protein, 18 aa, Artificial Sequence
  Hypothetical protein
PAGASRLLLLTGEIIDLP

SEQ ID NO: 9 - protein, 32 aa, Homo sapiens
EFLIVIKSMAPRGFSCLLLLTSEIDLPVKRRA

SEQ ID NO: 10 - protein, 32 aa, Homo sapiens
EFLIVIKSMAPRGFSALLLLTSEIDLPVKRRA

SEQ ID NO: 11 - protein, 32 aa, Homo sapiens
EFLIVIKSMAPRGFSCLLLLTGEIDLPVKRRA

SEQ ID NO: 12 - protein, 32 aa, Homo sapiens
EFLIVIKSMAPAGASCLLLLTGEIDLPVKRRA

SEQ ID NO: 13 - protein, 27 aa, Artificial Sequence
  Hybrid peptide (Colivelin)
SALLRSPIPAPAGASRLLLLTGEIDLP

SEQ ID NO: 14 - protein, 24 aa, Homo sapiens
MARRGFSCLLLSTTATDLPVKRRT

SEQ ID NO: 15 - protein, 24 aa, Homo sapiens
MAPRGASCLLLLTSEIDLPVKRRA

SEQ ID NO: 16 - protein, 24 aa, Homo sapiens
MAPRGASCLLLLTGEIDLPVKRRA

SEQ ID NO: 17 - protein, 24 aa, Homo sapiens
MAPRGASCLLLLTGEIDLPVARRA

SEQ ID NO: 18 - protein, 24 aa, Homo sapiens
MAKRGLNCLPHQVSEIDLSVQKRI

(19) United States
(12) Patent Application Publication
(10) Pub. No.: US 2013/0123168 A1
(43) Pub. Date: May 16, 2013

(54) HUMANIN AND HUMANIN-ANALOGUES FOR THE MANAGEMENT OF ATHEROSCLEROSIS

(71) Applicants: Pinchas Cohen, Pacific Palisades, CA (US); Amir Lerman, Rochester, MN (US); Lilach O. Lerman, Rochester, MN (US)

(72) Inventors: Pinchas Cohen, Pacific Palisades, CA (US); Amir Lerman, Rochester, MN (US); Lilach O. Lerman, Rochester, MN (US)

(73) Assignee: The Regents of the University of California, Oakland, CA (US)

(21) Appl. No.: 13/526,309

(22) Filed: Jun. 18, 2012

Related U.S. Application Data
(60) Provisional application No. 61/498,474, filed on Jun. 17, 2011.

(51) Int. Cl. A61K 38/17; A61K 38/10

(52) U.S. Cl. CPC A61K 38/1709; A61K 38/10
USPC 514/1.9; 514/44 R; 514/21.4; 435/375; 73/61.43

(57) ABSTRACT

The present invention is directed to methods and compositions for preventing or reducing atherosclerotic lesions and plaques. Specifically, the instant invention provides methods for using humanin and its analogues to prevent or reduce the formation of atherosclerotic plaques. Also provided are methods of using humanin and its analogues to improve the survival of endothelial cells, particularly aortic endothelial cells.

### CROSS-REFERENCES TO RELATED APPLICATIONS

The present application claims priority to U.S. Ser. No. 61/498,474, filed Jun. 17, 2011, herein incorporated by reference in its entirety.

### STATEMENT OF GOVERNMENT RIGHTS

This invention was made with Government support under Grant Nos. AG034430 and GM090311, awarded by the National Institutes of Health. The Government has certain rights in the invention.

### INTRODUCTION

Cardiovascular disease is a leading cause of morbidity and mortality, particularly in the United States and in Western European countries. Several causative factors are implicated in the development of cardiovascular disease including hereditary predisposition to the disease, gender, lifestyle factors such as smoking and diet, age, hypertension, and hyperlipidemia, including hypercholesterolemia.

Atherosclerosis is the most prevalent of cardiovascular diseases. Atherosclerosis is the principal cause of heart attack, stroke, and gangrene of the extremities and as such, the principle cause of death in the United States.

Atherosclerosis is a general term for the thickening and hardening of arteries. Arteries comprise three main layers. The outside layer (the external elastic lamina or the adventitia) supports the artery and is composed predominantly of loose connective tissue. The middle layer (between the lamina elastica interna and externa), comprises predominantly smooth muscle (in mice this layer is very thin: 1-2 cells). The muscle cells provide for contraction and relaxation of the artery which controls the rate of blood flow. The inner layer of the artery is itself composed of three layers: an elastic layer (the internal elastic lamina), a basement layer (the intima) and an innermost layer (the endothelium).

Endothelial dysfunction is an important step in the development of atherosclerosis. Endothelial cells, smooth muscle cells, and macrophages produce reactive oxygen species (ROS). ROS oxidize low-density lipoprotein (LDL) to form oxidized-LDL (Ox-LDL). Ox-LDL causes vascular smooth muscle cells, endothelial cells and macrophages to undergo apoptosis. The resulting cellular debris, in combination with lipids, cholesterol, and calcium, form plaque.

Humanin (HN) is a 24 amino acid peptide originally isolated from a cDNA library of surviving neurons of familial Alzheimer's disease (AD) and is expressed from an open reading frame within the mitochondrial 16S ribosomal RNA. HN transcripts are present in kidney, testis, brain, and the gastrointestinal tract. HN is also highly conserved among species, being identified in plants, nematodes, rats, mice, and many other species, demonstrating that it is highly conserved along evolution (Guo et al., 2003).

Research indicates that HN is cytoprotective, functioning as a wide spectrum survival factor (Nishimoto et al., Trends Mol. Med., 10:102-5 (2004)). HN is known as a rescue factor against neuronal cell death associated with AD. And HN has also been shown to prevent pancreatic beta cell apoptosis in a diabetes model. The mechansim by which HN mediates this cytoprotective affect is unclear.

In addition to HN, HN analogues are known. These HN analogues are described as possessing enhanced cytoprotective properties. These analogs include HNG (S14G) (Hashimoto et al., J. Neurosci., 21: 9235-9245 (2001) and Terashita et al., J. Neurochem., 85: 1521-1538 (2003)), HNG-F6A (Ikonen et al., Proc Nat Acad. Sci., 100: 13042-13047 (2003)) and colivelin. HN and its analogues and derivatives have shown therapeutic potential for an array of diseases including Alzheimer's disease, diabetes and kidney failure.

There remains a need for new methods and compositions for alleviating the symptoms associated with atherosclerosis including the formation of plaque. The instant invention addresses this and other needs by providing methods of using HN and HN analogues in preventing and/or treating atherosclerosis, particularly atherosclerotic plaques and lesions.

### BRIEF DESCRIPTION OF THE DRAWINGS

FIG. 1 depicts the affects of humanin on reactive oxygen species production by human aortic endothelial cells (HAEC). To test whether humanin (HN) has any affect on induced reactive oxygen species (ROS) production in HAEC, the fluorescent probe dihydroethidium (DHE) was used to detect the presence of superoxide in living cells. (A) DHE fluorescence of HAECs preincubated with or without HN overnight and then exposed to oxidize low density lipoprotein (Ox-LDL) to trigger ROS formation. Flourescent images of 10-12 non-overlapping fields taken at low magnification (15×). Bar, 100 μm. (B) Comparison of pretreatment with HN or irrelevant peptide prior to Ox-LDL exposure photographed at 90× magnification. (C) HN titration effects were measured. DHE fluorescence is expressed as average relative fluorescent units (n≧100 cells/experimental condition)

FIG. 2 depicts the affects of HN on ROS induced apoptosis of HAEC. TUNEL (Terminal deoxynucleotidyl transferase dUTP nick end labeling) assay was used to detect apoptosis among HAECs. Nuclei were visualized by DAPI staining and cells were imaged by epifluorescence microscopy. (A) Comparison of fluorescence detected by TUNEL between cultured HAECs in the presence of Ox-LDL and with or without HN. Upper panels: HAECs treated with Ox-LDL without HN preincubation; Lower panels: cells pretreated overnight with HN followed by Ox-LDL incubation. Bar 100 μm. (B) Graph summarizing TUNEL assay results under indicated culture conditions.

FIG. 3 depicts the affects of HN on cellular ceramide levels. Long chain ceramides are thought to be involved in apoptosis. HAECs were treated overnight with or without HN and then incubated in the presence of Ox-LDL. Results of detected ceramides are shown graphically.

FIG. 4 depicts the affects of the HN analogue HNGF6A on the proximal aorta of animals. The percent relaxation of proximal aorta isolated from four different groups of animals was examined in the presence of acetylcholine (A), nitroprusside (B), or the calcium ionophore A23187 (C). Group 1: C57Bl/6 mice normal diet/intraperitoneal (IP) saline; Group 2: C57Bl/6 mice normal diet/IP HNGF6A; Group 3: Apo E deficient mice high cholesterol diet/IP saline; Group 4: Apo E deficient mice high cholesterol diet/IP HNGF6A.

FIG. 5 depicts histological and morphometric analyses of proximal aorta cross-sections. Plaque size among mice of Group 4 (0.01±0.003 mm²) was significantly larger than that of Group 3 (0.9±0.01 mm²), while no plaque was apparent in the aortas from Groups 1 and 2. (A) Group 3; (B) Group 4 (C) Graphical comparison of results from Group 3 and Group 4.

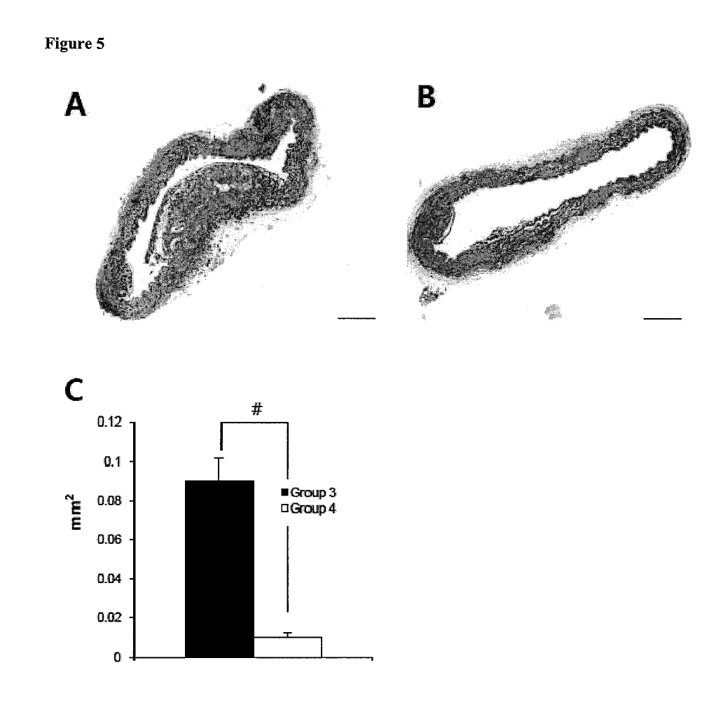

FIG. 6 depicts TUNEL staining in the aortic arch from Group 3 (A) and Group 4 (B) mice. HNGF6A treatment to ApoE-deficient mice on a high cholesterol diet showed significantly decreased numbers of apoptotic cells in the examined plaques compared to Group 3 mice (ApoE-deficient mice on a high cholesterol diet). (C) graphical comparison of the results.

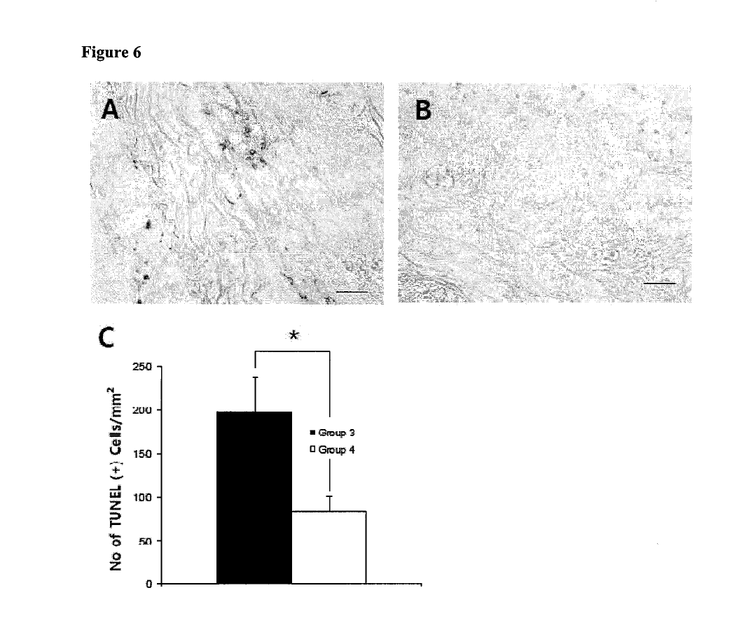

FIG. 7 shows the results from an analysis of plasma adipokine and cytokine levels from each group of mice. t-PA-1 and VEGF demonstrated statistically significant differences between Group 1 and Groups 3 and 4. However, there were no observed differences in the levels of adipokines and cytokines between Group 3 and Group 4; P, 0.05 relative to Group 1.

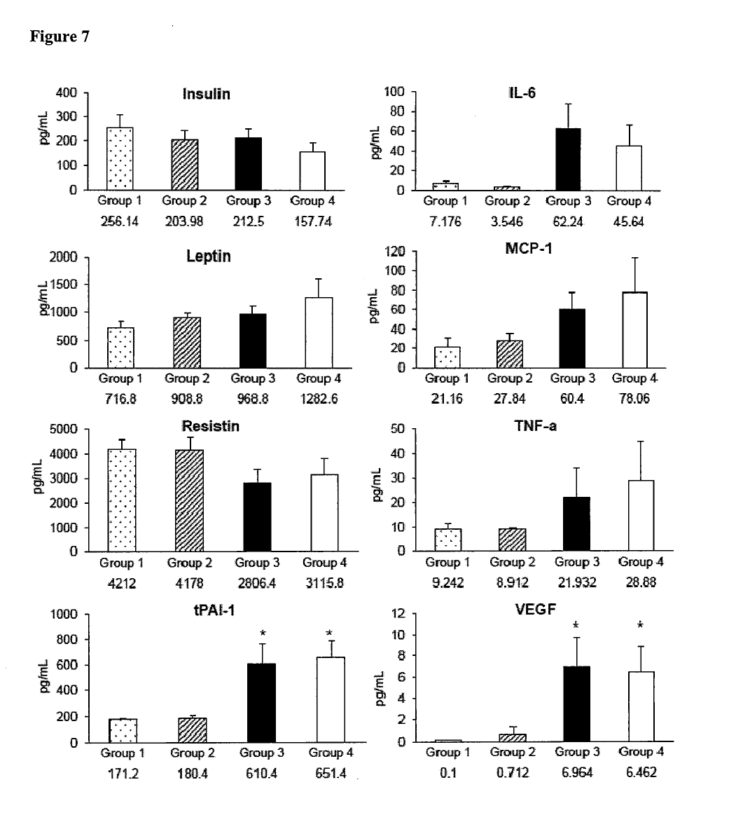

FIG. 8 depicts the results from the analysis of plasma HN levels detected in patients with and with coronary endothelial dysfunction. Plasma HN level was significantly lower in patients with coronary endothelial dysfunction relative to those patients with normal coronary endothelial function.

### BRIEF SUMMARY OF THE INVENTION

The invention is directed to methods for ameliorating symptoms and disease associated with endothelial dysfunction, atherosclerosis, atherosclerotic plaques and improving the survival of endothelial cells.

In some embodiments, methods for preventing or inhibiting the progression of atherosclerosis in a subject are provided, these methods encompass administering to a subject a composition comprising humanin or a humanin analogue in an amount effective to prevent or inhibit the progression of atherosclerosis as compared to a subject untreated with humanin or a humanin analogue.

In some embodiments, methods for inhibiting the progression of atherosclerosis in a subject encompasses administering to a subject a composition comprising humanin or a humanin analogue in an amount effective to reduce atherosclerotic plaque size as compared to a subject untreated with humanin or a humanin analogue.

In some embodiments, methods for treating atherosclerosis are provided the method encompassing the administration of a composition containing humanin or a humanin analogue to a subject so as to reduce the size of fibrotheroma(s) as compared to a subject untreated with humanin or a humanin analogue. In other embodiments, methods for treating atherosclerosis are provided the methods encompassing the administration of a composition containing humanin or a humanin analogue to a subject so as to reduce the size of atheroma(s) as compared to a subject untreated with humanin or a humanin analogue. In some embodiments, methods for treating atherosclerosis are provided the methods encompassing the administration of a composition containing humanin or a humanin analogue to a subject so as to reduce the size of intermediate lesion(s) as compared to a subject untreated with humanin or a humanin analogue.

In some embodiments, methods for treating atherosclerosis are provided the methods encompassing the administration of a composition containing humanin or a humanin analogue to a subject prior to the formation of detectable fibrotheroma(s). In other embodiments, methods for treating atherosclerosis are provided, the method encompassing the administration of a composition containing humanin or a humanin analogue to a subject prior to the formation of detectable atheroma(s). In other embodiments, methods for treating atherosclerosis are provided, the methods encompassing the administration of a composition containing humanin or a humanin analogue to a subject prior to the formation of detectable intermediate lesion(s).

In some embodiments, the subject is a mammal. In other embodiments, the subject is a human.

In some embodiments, the subject does not have diabetes. In other embodiments, the subject has not had a myocardial infarction or myocardial injury. In still other embodiments, the subject has not had myocardial ischemia.

In some embodiments, methods for improving the survival of endothelial cells are provided, the methods encompassing contacting endothelial cells with a composition comprising humanin or a humanin analogue in an amount effective to improve survival of the endothelial cells as compared to an untreated population of endothelial cells. In some embodiments, the endothelial cells are in vitro.

In some embodiments, the humanin analogue is selected from the group consisting of: S14G-HN; C8A-HN, D-Ser14-HN; AGA-HNG; AGA-(D-Ser14)-HN; AGA-(D-Ser14)-HN17; AGA-(C8R)-HNG17; EF-HN; EF-HNA; EF-HNG; EF-AGA-HNG; colivelin; P3R HN; F6A-HN; F6A-HNG; F6AK21A-HNG; and Z-HN. In some embodiments, methods for treating atherosclerosis are provided, the methods encompassing the administration of a composition containing humanin or a humanin analogue. In some embodiments, the humanin analogue is F6A-HNG (SEQ ID NO:16). In other embodiments, the disclosed methods encompass the administration of more than one humanin analogue, or humanin in combination with at least one humanin analogue.

In some embodiments, HN or HN analogue(s) are administered in combination with a second therapeutic. In some embodiments, the second therapeutic is a lipid lowering drug. In some embodiments, HN or HN analogue(s) are administered at the same time as the second therapeutic composition. In some embodiments, HN or HN analogue(s) are administered separate from the second therapeutic composition, for example, on an independent dosing schedule.

In some embodiments, methods of treating atherosclerosis are provided, the methods encompassing the administration of humanin or a humanin analog whereby the humanin or humanin analogue is administered by administering a vector encoding the humanin or humanin analogue to the subject such that the humanin or humanin analogue is expressed from the vector.

In some embodiments, methods for measuring endothelial function in a subject are provided, the methods encompassing measuring HN concentration from a bodily of a subject, comparing the concentration of HN in the bodily fluid from the subject with that of control subject or value, and based on the results of the comparison starting a treatment regimen. In some embodiments, the treatment regimen encompasses administering humanin or a humanin analogue.

### DETAILED DESCRIPTION OF THE INVENTION

### A. Introduction

Atherosclerosis is a complex disease involving many cell types and molecular factors. Atherosclerosis occurs in response to insults to the endothelium leading to the formation of plaques. Such plaques occlude the blood vessels and thus restrict the flow of blood, resulting in a lack of oxygen supply to tissues due to inadequate perfusion.

Pathologic conditions associated with atherosclerotic plaque formation include for example, atherosclerosis, stroke, heart attacks, unstable angina and gangrene due to blocked blood vessels.

During the early stages of atherosclerosis, the endothelial cell layer in the arterial vessel is damaged resulting in the penetration of low density lipoprotein (LDL) into the subendothelial space. Oxidation of the LDL (Ox-LDL) by reactive oxygen species released by endothelial cells acts to amplify this process resulting in enhanced oxidative stress, causing cell death, ultimately resulting in the formation of atherosclerotic plaques.

Thus, endothelial dysfunction is a marker of the atherosclerosis process and is accompanied by a proinflammatory, proliferative and procoagulatory milieu. Increased oxidative stress is one of the mechanisms of endothelial dysfunction.

Humanin (HN) is a polypeptide defined by the sequence of SEQ ID NO:1: MAPRGFSCLLLLTSEIDLPVKRRA. Humanin activities include IGFBP-3 binding; inducing cell signaling and STAT-3 activation; reducing apoptosis of neuronal cells; and improving survival of pancreatic beta islet cells.

The exact mechanism of humanin's protective activity and interaction with IGFBP-3 may rely on: a) dimerization (Terashita et al. (2003) J Neurochem. 85:1521-38); b) FPRL-1 binding (Guo et al. (2003) Nature 423:456-61); c) tyrosine kinase activation (lung and Van Nostrand (2003) J. Neurochem. 84:266-72), d) STAT-3 activation (Maximov et al. (2002) Med Hypotheses 59:670-73); and e) antagonism of the pro-apoptotic molecules BimEL and Bid (Caricasole et al. (2002) FASEB J 16:1331-33; Tajima et al. (2002) Neurosci Lett. 324:227-31). In addition, TRIM11 plays a role in the regulation of intracellular humanin levels through ubiquitin-mediated protein degradation pathways.

This invention relates to methods for preventing or inhibiting the progression of a pathologic condition associated with atherosclerotic plaque formation. This invention entails the administration of compositions composed of humanin or humanin analogues.

### B. Definitions

As used herein, “atherosclerosis” (also known as arteriosclerotic vascular disease or ASVD) refers to a condition in which arterial walls thicken. Atherosclerosis progresses over the course of years and even decades. The progression of atherosclerosis can be divided into six general progressive states: initial lesion, fatty streak, intermediate lesion, atheroma (or atherosclerotic plaques), fibroatheroma, and complicated lesion.

“Humanin” is a secreted peptide defined by the 24 amino acid sequence of SEQ ID NO:1: MAPRGFSCLLLLTSEIDLPVKRRA. Humain also includes substantially similar peptides and analogues, as defined herein. Humanin activities include IGFBP-3 binding; inducing cell signaling and STAT-3 activation; reducing atherosclerotic plaques; and improving survival of endothelial cells.

“Humanin analogues,” “humanin derivatives,” and equivalent terms, refer to peptides with at least one humanin activity. One of skill can determine whether any particular peptide is a humanin analogue, for example, by determining whether the peptide is capable of neuroprotection in an established humanin assay, e.g., as described in Chiba et al. (2005) J. Neuroscience 25:10252-61; or by determining whether the peptide is capable as a survival factor for neuroendocrine beta cells, e.g., as described in US 2010/0130412.

Generally, the humanin analogue comprises 17-50 amino acids comprising the amino acid sequence of SEQ ID NO:19. As used herein, an amino acid sequence providing the designation (x/y), as in SEQ ID NO:19, indicates that either amino acid x or amino acid y can be used at the indicated position. Analogues include, but are not limited to those shown in Table 1.

TABLE 1
Humanin analogues

| | SEQ ID | |
|---|---|---|
| NAME | NO | SEQUENCE |
| humanin | 1 | MAPRGFSCLLLLTSEIDLPVKRRA |
| S14G-HN (HNG) | 2 | MAPRGFSCLLLLTGEIDLPVKRRA |
| C8A-HN (HNA) | 3 | MAPRGFSALLLLTSEIDLPVKRRA |
| D-Ser14-HN | 4 | MAPAGASCLLLLTS*EIDLPVKRRA |
| AGA-HNG | 5 | MAPAGASCLLLLTGEIDLPVKRRA |
| AGA-(D-Ser14) | 6 | MAPAGASCLLLLTS*EIDLPVKRRA |
| AGA-(D-Ser14)- | 7 | PAGASCLLLLTS*EIDLP |
| HN17 | | |
| AGA-(C8R)- | 8 | PAGASRLLLLTGEIIDLP |
| HNG17 | | |
| EF-HN | 9 | EFLIVIKSMAPRGFSCLLLLTSEIDLPVKRRA |
| EF-HNA | 10 | EFLIVIKSMAPRGFSALLLLTSEIDLPVKRRA |
| EF-HNG | 11 | EFLIVIKSMAPRGFSCLLLLTGEIDLPVKRRA |
| EF-AGA-HNG | 12 | EFLIVIKSMAPAGASCLLLLTGEIDLPVKRRA |
| Colivelin | 13 | SALLRSPIPA-PAGASRLLLLTGEIDLP |
| P3R-HN | 14 | MARRGFSCLLLSTTATDLPVKRRT |
| F6A-HN | 15 | MAPRGASCLLLLTSEIDLPVKRRA |
| HNGF6A | 16 | MAPRGASCLLLLTGEIDLPVKRRA |
| F6AK21A | 17 | MAPRGASCLLLLTGEIDLPVARRA |
| Z-HN | 18 | MAKRGLNCLPHQVSEIDLSVQKRI |
| Consensus | 19 | (P/R/A)(R/A/G)(G/A)(F/A)S(C/R) sequence |
| | | LLL(L/S)T(S/T/G)(E/A)(I/T)DLP |

S* indicates D-serine

Some of the humanin analogues have increased potency compared to humanin, or slightly altered activities. Z-FIN (SEQ ID NO:18) promotes survival and activates STAT-3 and ERK in NIT cells with a two-fold greater potency than humanin. F6AK21A-HNG (SEQ ID NO: 17) and HNGF6A (SEQ ID NO:16) demonstrate similar activities with even greater potency.

“Endothelial cells” refer to cells that make up the endothelium. Endothelial cells line the interior surface of blood vessels and lymphatic vessels, thus forming an interface between circulating blood and lymph and the vessel wall.

“Bodily fluid” refers to a naturally occurring fluid from an animla, such as saliva, sputum, serum, plasma, blood, urine, mucus, gastric juices, pancreatic juices, semen, products of lactation or menstruation, tears, or lymph.

As used herein, “improving cell survival” or “improving the survival of endothelial cells” refers to an increase in the number of cells that survive a given condition, as compared to a control, e.g., the number of cells that would survive the same conditions in the absence of treatment. Conditions can be in vitro, in vivo, ex vivo, or in situ. Improved cell survival can be expressed as a comparative value, e.g., twice as many cells survive if cell survival is improved two-fold. Improved cell survival can result from a reduction in apoptosis, an increase in the life-span of the cell, or an improvement of cellular function and condition. In some embodiments, cell survival is improved by 5, 10, 20, 30, 40, 50, 60, 70, 80, 90 or 100%, as compared to control levels. In some embodiments, cell survival is by two-, three-, four-, five-, or ten-fold of control levels. Alternatively, improved cell survival can be expressed as a percentage decrease in apoptosis. In some embodiments, for example, apoptosis is reduced by 10, 20, 30, 40, 50, 60, 70, 80, 90 or up to 100%, as compared to a control sample.

The methods of this invention also relate to inhibiting, reducing, slowing or preventing the progression of atherosclerosis in a subject.

For example, as used herein, “reducing” or grammatical equivalents thereof can refer to a difference in size of atherosclerotic plaques observed between those subjects treated with humanin or humanin analogues and those untreated. That is, plaques are relatively smaller by 5%, 10%, 15%, 20%, 25%, 30%, 35%, 40%, 45%, 50%, 55%, 60%, 65%, 70%, 75%, 80%, 85%, 90%, 95% or more between treated as compared to untreated subjects. Further, reduce may also refer to a relative reduction in the percentage or numbers of cells observed undergoing apoptosis. A reduction is observed if the number or percentage of observed cells undergoing apoptosis in the humanin or humanin analogue treated subject is 95%, 90%, 85%, 80%, 75%, 70%, 65%, 60%, 55%, 50%, 45%, 40%, 35%, 30%, 25%, 20%, 15%, 10%, 5%, or less relative to untreated with humanin or humanin analogue. A reduction can further refer to the reduction in the progression of atherosclerosis or a reduction of endothelial dysfunction as described herein.

The term “preventing” as used herein is not intended as an absolute term. Instead, prevention refers to the delay of onset or reduced severity of symptoms associated with the disorder. In some circumstances, the symptoms of a subject receiving the compositions of the invention are 90%, 80%, 70%, 60%, 50%, 40%, 30%, 20%, 10%, 5%, 1% or less as compared to symptoms experienced by an untreated individual.

Similarly, the term “treating” is not intended to be an absolute term. In some circumstances, treatment leads to an improved prognosis or a reduction in the frequency or severity of symptoms.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologus protein indicates that the protein comprises two or more subsequences that are not found in the same relationship to each other in nature (e.g., a fusion protein).

The term “isolated” as used herein with respect to nucleic acids, such as DNA or RNA, refers to molecules separated from other DNAs, or RNAs, respectively that are present in the natural source of the macromolecule. Isolated is meant to include nucleic acid fragments which are not naturally occurring as fragments and would not be found in the natural state. The term isolated as used herein also refers to a nucleic acid or peptide that is substantially free of cellular material, viral material, or culture medium when produced by recombinant DNA techniques, or chemical precursors, or other chemicals when chemically synthesized.

The term “nucleic acid” refers to deoxyribonucleotides or ribonucleotides and polymers thereof in either single- or double-stranded form, and complements thereof. The term refers to all forms of nucleic acids (e.g., gene, pre-mRNA, mRNA) and their polymorphic variants, alleles, mutants, and interspecies homologs. The term nucleic acid is used interchangeably with gene, cDNA, mRNA, oligonucleotide, and polynucleotide. The term encompasses nucleic acids that are naturally occurring or recombinant. Nucleic acids can (1) code for an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence.

A particular nucleic acid sequence also implicitly encompasses “splice variants” and nucleic acid sequences encoding truncated forms of a protein. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N terminal end or the C-terminal end. Truncated versions of nucleic acid or polypeptide sequences can be naturally occurring or recombinantly created.

Nucleic acids can contain known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M). See, e.g., Creighton, Proteins (1984).

The term “identical” or “identity” or “percent identity,” or “sequence identity” in the context of two or more nucleic acids or polypeptide sequences that correspond to each other refer to two or more sequences or subsequences that are the same or have a specified percentage of amino acid residues or nucleotides that are the same (e.g., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical” and are embraced by the term “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists for a specified entire sequence or a specified portion thereof or over a region of the sequence that is at least about 25 amino acids or nucleotides in length, or more preferably over a region that is 50-100 amino acids or nucleotides in length. A corresponding region is any region within the reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted (e.g., by the local homology algorithm of Smith & Waterman, Adv. AppL Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

### C. Expression and Purification of Polypeptides

Naturally-occurring, synthetic, or recombinant polypeptides of the invention can be purified for use in compositions and functional assays. Naturally-occurring polypeptides of the invention can be purified from any source. Recombinant polypeptides can be purified from any suitable expression system (e.g., mammalian, insect, yeast, or bacterial cell culture).

The peptides of the present invention (i.e., humanin and humanin analogues) may include both modified peptides and synthetic peptide analogues. Peptides maybe modified to improve formulation and storage properties, or to protect labile peptide bonds by incorporating non-peptidic structures. Peptides of the present invention may be preparedusing methods known in the art. For example, peptides may be produced by chemical synthesis, e.g., using solid phase techniques and/or automated peptide synthesizers. In certain instances, peptides may be synthesized using solid phase strategies on an automated multiple peptide synthesizer (Abimed AMS 422) using 9-fluorenylmethyloxycarbonyl (Fmoc) chemistry. The peptides can then be purified by reversed phase-HPLC and lyophilized.

For recombinant approaches, the present invention includes isolated nucleic acids encoding the polypeptides disclosed herein, expression vectors comprising the nucleic acids, and host cells comprising the expression vectors. More particularly, the invention provides isolated nucleic acids encoding humanin peptides and humanin peptide analogues having humanin activities, the peptides including, but not limited to, the peptides having a sequence selected from the group consisting of SEQ ID NOS:1-19.

When recombinant proteins are expressed by the transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, but not limited to, by incubation in a buffer of about 100-150 ng/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook et al., both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques.

Alternatively, it is possible to purify proteins from bacteria periplasm. Where the protein is exported into the periplasm of the bacteria, the periplasmic fraction of the bacteria 10 can be isolated by cold osmotic shock in addition to other methods known to those of skill in the art (see, Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO4 and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

The polypeptides of the invention may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., Current Protocols in Molecular Biology (1995 supplement); and Sambrook et al. MOLECULAR CLONING: A LABORATORY MANUAL, 2nd Ed., (1989)).

A number of procedures can be employed when polypeptides are being purified. For example, polypeptides can be purified using ion exchange or immunoaffinity columns.

Often as an initial step, and if the protein mixture is complex, an initial salt fractionation can separate many of the unwanted host cell proteins (or proteins derived from the cell culture media) from the recombinant protein of interest. The preferred salt is ammonium sulfate Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

The proteins of interest can also be separated from other proteins on the basis of their size, net surface charge, hydrophobicity and affinity for ligands. In addition, antibodies raised against proteins can be conjugated to column matrices and the proteins immunopurified. All of these methods are well known in the art.

Immunoaffinity chromatography using antibodies raised to a variety of affinity tags such as hemagglutinin (HA), FLAG, Xpress, Myc, hexahistidine, glutathione S transferase (GST) and the like can be used to purify polypeptides. The His tag will also act as a chelating agent for certain metals (e.g., Ni) and thus the metals can also be used to purify His-containing polypeptides. After purification, the tag is optionally removed by specific proteolytic cleavage.

It will be apparent to one of skill that chromatographic techniques can be performed at any scale and using equipment from many different manufacturers (e.g., Pharmacia Biotech).

### D. Cell Culture and In Vitro Applications

In some embodiments, the compositions of the present invention are used to improve the survival of endothelial cells in culture. Cells to be cultured include explants and primary and/or transformed cell cultures derived from patient tissues. Such methods are useful for maintaining and/or improving the viability of a donor source for transplant. In some cases, the population of endothelial cells is expanded in culture.

Methods of cell culture are well known in the art. See, e.g., Freshney et al., Culture of Animal Cells, A Manual of Basic Technique (3rd ed. 1994), and the references cited therein for a discussion of cell culture conditions and how to isolate and culture cells from patients. In some embodiments, the cultured cells are initially undifferentiated or partially differentiated.

This aspect of the present invention relies upon routine techniques in the field of cell culture. In general, the cell culture environment includes consideration of such factors as the substrate for cell growth, cell density and cell contract, the gas phase, the medium, and temperature.

Incubation of cells is generally performed under conditions known to be optimal for cell survival. Such conditions may include, for example, a temperature of approximately 37° C. and a humidified atmosphere containing approximately 5% CO₂. The duration of the incubation can vary widely, depending on the desired results. Proliferation is conveniently determined using ³H thymidine incorporation or BrdU labeling.

Plastic dishes, flasks, or roller bottles may be used to culture cells according to the methods of the present invention. Suitable culture vessels include, for example, multi-well plates, Petri dishes, tissue culture tubes, flasks, roller bottles, and the like.

Cells are grown at optimal densities that are determined empirically based on the cell type. Cultured cells are normally grown in an incubator that provides a suitable temperature, e.g., the body temperature of the animal from which is the cells were obtained, accounting for regional variations in temperature. Generally, 37° C. is the preferred temperature for cell culture. Most incubators are humidified to approximately atmospheric conditions.

Defined cell media are available as packaged, premixed powders or presterilized solutions. Examples of commonly used media include MEM-a, DME, RPMI 1640, DMEM, Iscove's complete media, or McCoy's Medium (see, e.g., GibcoBRL/Life Technologies Catalogue and Reference Guide; Sigma Catalogue). Typically, MEM-a or DMEM are used in the methods of the invention. Defined cell culture media are often supplemented with 5-20% serum, typically heat inactivated serum. The culture medium is usually buffered to maintain the cells at a pH preferably from about 7.2 to about 7.4. Other supplements to the media typically include, e.g., antibiotics, amino acids, and sugars, and growth factors.

### E. Pharmaceutical Compositions

Pharmaceutical compositions and vaccines within the scope of the present invention can also contain other compounds, which can be biologically active or inactive. For example, one or more immunogenic portions of other antigens can be present, either incorporated into a fusion polypeptide or as a separate compound, within the composition or vaccine. Polypeptides can, but need not be, conjugated to other macromolecules as described, for example, within U.S. Pat. Nos. 4,372,945 and 4,474,757. Pharmaceutical compositions and vaccines can generally be used for prophylactic and therapeutic purposes.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, can be made into aerosol formulations (e.g., they can be “nebulized”) to be administered via inhalation. Aerosol formulations can be placed into pressurized acceptable propellants, such as dichlorodifluoromethane, propane, nitrogen, and the like.

Formulations suitable for parenteral administration, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration and intravenous administration are the preferred methods of administration. The formulations of commends can be presented in unit-dose or multi-dose sealed containers, such as ampules and vials.

Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention can be formulated as a lyophilizate. Compounds can also be encapsulated within liposomes using well known technology.

Injection solutions and suspensions can be prepared from sterile powders, granules, and tablets of the kind previously described. Cells transduced by nucleic acids for ex vivo therapy can also be administered intravenously or parenterally as described above.

The dose administered to a patient, in the context of the present invention should be sufficient to affect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17^{th} ed., 1989). Administration can be in any convenient manner, e.g., by injection, oral administration, inhalation, transdermal application, or rectal administration.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

Pharmaceutical and vaccine compositions can be presented in unit-dose or multi-dose containers, such as sealed ampoules or vials. Such containers are preferably hermetically sealed to preserve sterility of the formulation until use. In general, formulations can be stored as suspensions, solutions or emulsions in oily or aqueous vehicles. Alternatively, a vaccine or pharmaceutical composition can be stored in a freeze-dried condition requiring only the addition of a sterile liquid carrier immediately prior to use.

### F. Methods of Administration

Administration of the polypeptides of the present invention with a suitable pharmaceutical excipient as necessary can be carried out via any of the accepted modes of administration. Thus, administration can be, for example, intravenous, topical, subcutaneous, transcutaneous, transdermal, intramuscular, oral, intra joint, parenteral, intra-arteriole, intradermal, intraventricular, intracranial, intraperitoneal, intralesional, intranasal, rectal, vaginal, or by inhalation. Administration can be targeted directly to pancreatic tissue, e.g., via injection.

The compositions of the invention may be administered repeatedly, e.g., at least 2, 3, 4, 5, 6, 7, 8, or more times, or the composition may be administered by continuous infusion. Suitable sites of administration include, but are not limited to, dermal, mucosal, bronchial, gastrointestinal, anal, vaginal, eye, and ear. The formulations may take the form of solid, semi-solid, lyophilized powder, or liquid dosage forms, such as, for example, tablets, pills, lozenges, capsules, powders, solutions, suspensions, emulsions, suppositories, retention enemas, creams, ointments, lotions, gels, aerosols, or the like, preferably in unit dosage forms suitable for simple administration of precise dosages.

The term “unit dosage form” refers to physically discrete units suitable as unitary dosages for human subjects, each unit containing a predetermined quantity of active material calculated to produce the desired onset, tolerability, and/or therapeutic effects, in association with a suitable pharmaceutical excipient (e.g., an ampoule). In addition, more concentrated compositions may be prepared, from which the more dilute unit dosage compositions may then be produced. The more concentrated compositions thus will contain substantially more than, e.g., at least 1, 2, 3, 4, 5, 6, 7, 8, 9, 10, or more times the amount of a conjugate or a combination of conjugates.

Methods for preparing such dosage forms are known to those skilled in the art (see, for example, REMINGTON'S PHARMACEUTICAL SCIENCES, 18^{th} Ed., Mack Publishing Co., Easton, Pa. (1990). The composition to be administered contains a quantity of the peptides of the invention in a pharmaceutically effective amount for improving beta islet cell survival. In addition, pharmaceutically acceptable salts of the peptides of the present invention (e.g., acid addition salts) may be prepared and included in the compositions using standard procedures known to those skilled in the art of synthetic organic chemistry and described, e.g., by March, Advanced Organic Chemistry: Reactions, Mechanisms and Structure, 4^{th} Ed., New York, Wiley-Interscience (1992).

In another approach, nucleic acids encoding the polypeptides of the invention are used for transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acids, under the control of a promoter, then express a polypeptide of the present invention, thereby mitigating the effects of a disease associated with reduced insulin production.

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies (for a review of gene therapy procedures, see Anderson, Science, 256:808-813 (1992); Nabel et al., TIBTECH, 11:211-217 (1993); Mitani et al., TIBTECH, 11:162-166 (1993); Mulligan, Science, 926-932 (1993); Dillon, TIBTECH, 11:167-175 (1993); Miller, Nature, 357:455-460 (1992); Van Brunt, Biotechnology, 6(10):1149-1154 (1998); Vigne, Restorative Neurology and Neuroscience, 8:35-36 (1995); Kremer et al., British Medical Bulletin, 51(1):31-44 (1995); Haddada et al., in Current Topics in Microbiology and Immunology (Doerfler & Bohm eds., 1995); and Yu et al., Gene Therapy, 1:13-26 (1994)).

For delivery of nucleic acids, viral vectors may be used. Suitable vectors include, for example, herpes simplex virus vectors as described in Lilley et al., Curr. Gene Ther., 1(4):339-58 (2001), alphavirus DNA and particle replicons as described in e.g., Polo et al., Dev. Biol. (Basel), 104:181-5 (2000), Epstein-Barr virus (EBV)-based plasmid vectors as described in, e.g., Mazda, Curr. Gene Ther., 2(3):379-92 (2002), EBV replicon vector systems as described in e.g., Otomo et al., J. Gene Med., 3(4):345-52 (2001), adeno-virus associated viruses from rhesus monkeys as described in e.g., Gao et al., PNAS USA., 99(18):11854 (2002), adenoviral and adeno-associated viral vectors as described in, e.g., Nicklin et al., Curr. Gene Ther., 2(3):273-93 (2002). Other suitable adeno-associated virus (AAV) vector systems can be readily constructed using techniques well known in the art (see, e.g., U.S. Pat. Nos. 5,173,414 and 5,139,941; PCT Publication Nos. WO 92/01070 and WO 93/03769; Lebkowski et al., Mol. Cell. Biol., 8:3988-3996 (1988); Vincent et al. (1990) Vaccines 90 (Cold Spring Harbor Laboratory Press); Carter, Current Opinion in Biotechnology 3:533-539 (1992); Muzyczka, Current Topics in Microbiol. and Immunol., 158:97-129 (1992); Kotin, Human Gene Therapy, 5:793-801 (1994); Shelling et al., Gene Therapy, 1:165-169 (1994); and Zhou et al., J. Exp. Med., 179:1867-1875 (1994)). Additional suitable vectors include E1B gene-attenuated replicating adenoviruses described in, e.g., Kim et al., Cancer Gene Ther., 9(9):725-36 (2002) and nonreplicating adenovirus vectors described in e.g., Pascual et al., J. Immunol., 160(9):4465-72 (1998). Exemplary vectors can be constructed as disclosed by Okayama et al., Mol. Cell. Biol., 3:280 (1983).

Molecular conjugate vectors, such as the adenovirus chimeric vectors described in Michael et al., J. Biol. Chem., 268:6866-6869 (1993) and Wagner et al., Proc. Natl. Acad. Sci. USA, 89:6099-6103 (1992), can also be used for gene delivery according to the methods of the invention.

In one illustrative embodiment, retroviruses provide a convenient and effective platform for gene delivery systems. A selected nucleotide sequence encoding a polypeptide of the invention is inserted into a vector and packaged in retroviral particles using techniques known in the art. The recombinant virus can then be isolated and delivered to a subject. Suitable vectors include lentiviral vectors as described in e.g., Scherr et al., Curr. Gene Ther., 2(1):45-55 (2002). Additional illustrative retroviral systems have been described (e.g., U.S. Pat. No. 5,219,740; Miller et al., BioTechniques, 7:980-990 (1989); Miller, Human Gene Therapy, 1:5-14 (1990); Scarpa et al., Virology, 180:849-852 (1991); Burns et al., Proc. Natl. Acad. Sci. USA, 90:8033-8037 (1993); and Boris-Lawrie et al., Curr. Opin. Genet. Develop., 3:102-109 (1993).

Other known viral-based delivery systems are described in, e.g., Fisher-Hoch et al., Proc. Natl. Acad. Sci. USA, 86:317-321 (1989); Flexner et al., Ann. N.Y. Acad. Sci., 569:86-103 (1989); Flexner et al., Vaccine, 8:17-21 (1990); U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques, 6:616-627 (1988); Rosenfeld et al., Science, 252:431-434 (1991); Kolls et al., Proc. Natl. Acad. Sci. USA, 91:215-219 (1994); Kass-Eisler et al., Proc. Natl. Acad. Sci. USA, 90:11498-11502 (1993); Guzman et al., Circulation, 88:2838-2848 (1993); Guzman et al., Cir. Res., 73:1202-1207 (1993); and Lotze et al., Cancer Gene Ther., 9(8):692-9 (2002).

### G. Therapeutic and Prophylactic Applications

In certain aspects, the compositions of the invention are used for the treatment or prevention of a disease or disorder in a subject in need thereof. Examples of diseases or disorders suitable for treatment with the humanin or humanin analogue compositions described herein include, but are not limited to, those disorders characterized by atherosclerosis or atherosclerotic plaques, and endothelial cell death.

In some embodiments, methods for inhibiting or reducing the formation of an initial atherosclerotic lesion in a subject are provided, these methods encompass administering to a subject a composition comprising humanin or a humanin analogue in an amount effective to inhibit the formation of an initial atherosclerotic lesion as compared to a subject untreated with humanin or a humanin analogue. In other embodiments, methods for inhibiting the formation of atherosclerotic fatty streak(s) in a subject are provided, these methods encompass administering to a subject a composition comprising humanin or a humanin analogue in an amount effective to inhibit the formation of atherosclerotic fatty streak(s) as compared to a subject untreated with humanin or a humanin analogue.

The initial lesion appears “normal” histologically, however macrophage infiltration can be detected and foam cells can be isolated. The initial lesion then develops to a fatty streak in which lipids accumulate. The fatty streak is followed by the intermediate lesion, wherein intracellular lipids accumulate and small extracellular pools of lipids form.

Atheroma or atherosclerotic plaque follows the intermediate lesion and is characterized by the continued accumulation of intracellular lipid and the formation of a core of extracellular lipid. Subsequently, the atheroma becomes fibrotic, with single or multiple lipid cores and calcific and fibrous layers. Such a lesion is referred to as a fibroatheroma.

In other embodiments, a composition a method for inhibiting the formation of atheroma(s) in a subject are provided, this method encompasses administering to a subject a composition comprising humanin or a humanin analogue in an amount effective to inhibit the formation of atheroma(s) as compared to a subject untreated with humanin or a humanin analogue.

The final form of atherosclerotic lesion is the complicated lesion, which can be responsible for hematoma-hemorrhage and thrombosis. In some embodiments, methods for inhibiting the formation of a complicated lesion in a subject are provided, these methods encompass administering to a subject a composition comprising humanin or a humanin analogue in an amount effective to inhibit the formation of a complicated lesion as compared to a subject untreated with humanin or a humanin analogue.

The methods of this invention are applicable to any subject in need thereof. The subject in need thereof may be any mammal which has a predilection for developing atherosclerosis, for example a subject who has a family history of developing atherosclerotic plaques, a subject having Familial Hypercholesteremia, which is an inherited disorder that leads to high cholesterol levels, or a subject having high plasma cholesterol levels without a family history of high cholesterol, or any mammal already having atherosclerotic plaques in one or more arteries.

By inhibiting or reducing the initiation and progression of plaque formation, the initiation and progression of pathologic conditions associated with plaque formation, e.g., atherosclerosis, stroke, heart attacks, unstable angina and gangrene associated with a blocked blood vessel, are also inhibited. Those of skill in the art are well aware of methods used to determine if a subject harbors atherosclerotic plaques or has an increased chance of developing atherosclerotic plaques (see, e.g., Williams Hematology, 2^{d} Ed, Beutler et al. eds., (2001), chapter 30; Ross, N. Engl. J Med. 340, 115-126 (1999), Lusis, “Atherosclerosis.” Nature 407, 233-241 (2000) (all incorporated herein by reference). The atherosclerotic plaques may be end stage plaques, e.g., vulnerable plaques, unstable plaques or rupture prone plaques or any combination thereof.

This invention also provides a method of mitigating (e.g., reducing or eliminating) one or more symptoms of atherosclerosis in a mammal (human or non-human mammal). The method typically involves administering to the mammal an effective amount of humanin and/or one or more of the humanin analogues described. In certain embodiments, the mammal is a mammal diagnosed as having one or more symptoms of atherosclerosis. In certain embodiments, the mammal is a mammal diagnosed as at risk for stroke or atherosclerosis.

The methods described herein further include measuring endothelial function in a subject. The method comprises measuring the humanin concentration from the bodily fluid of a subject, and comparing the concentration of humanin in the bodily fluid from the subject with that of a control subject or value, and start a treatment regimen based on the results.

### H. Transplantation of Endothelial Cells

Another approach for treatment of atherosclerosis is transplant of endothelial tissue into an individual with reduced blood insulin levels. Cells for transplant are generally harvested from a donor individual or population of individuals that are distinct from the recipient (or host). Using these methods, immune suppression of the recipient is necessary to prevent immune rejection by the recipient. Given the unwanted side effects of immunosuppression, however, interest is growing in culturing the recipient's own cells for reintroduction.

Cells to be transplanted can be treated with the compositions of the invention before introduction into the host. Once the endothelial cells are transplanted, the compositions of the invention can be administered to the host systemically or directly to the site of transplantation, as described above.

Methods for culturing endothelial cells are described above. For reviews of transplant techniques, see, e.g., Claiborn and Stoffers (2008) Mt Sinai J Med 75:362-71; Eisenbarth (2007) J. Clin. Endocrinol. & Metabol. 92:2403-07; and references cited therein.

All publications and patent applications cited in this specification are herein incorporated by reference as if each individual publication or patent application were specifically and individually indicated to be incorporated by reference.

Although the invention is described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

### Example 1

### Humanin Attenuates Oxidized LDL Induced Reactive Oxygen Species Production in Human Aortic Endothelial Cells

Prior studies have reported that treatment of vascular endothelial cells with oxidized low density lipoprotein (LDL) stimulates reactive oxygen species (ROS) formation. To test whether humanin (HN) affects Ox-LDL induced ROS production in endothelial cells, the fluorescent probe dihydroethidium was utilized in order to detect superoxide in living cells. DHE fluorescence was measured in human aortic endothelial cells (HAECs) preincubated with HN and subsequently exposed to Ox-LDL. Intracellular ROS production was monitored by following the conversion of the oxidant sensitive dye dihydroethidine to fluorescent ethidium.

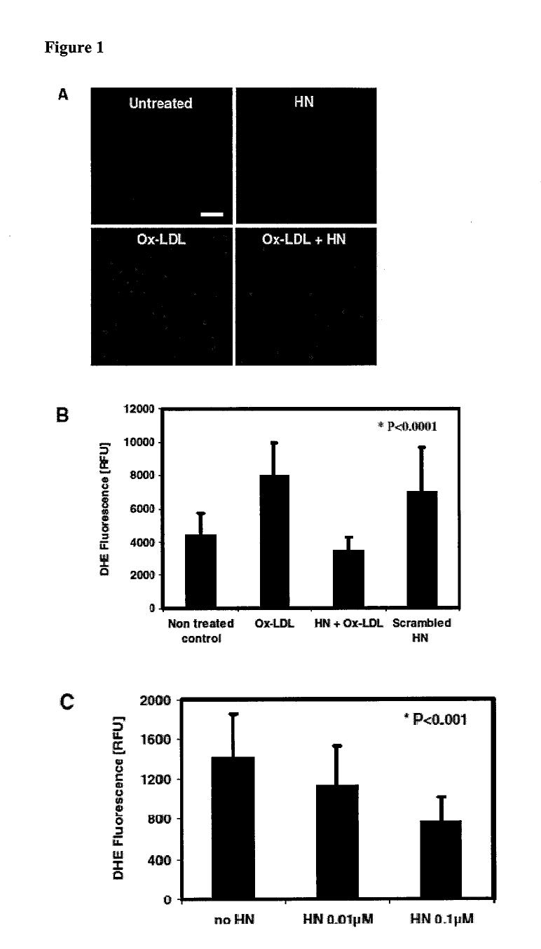

To accomplish this, as described in Bachar et al. (2010) Cardiovasc. Res. 88(2):360-6 (incorporated by reference in its entirety). HAECs were maintained in M200 medium containing low serum growth supplement and 1% penicillin-streptomycin (Invitrogen). HAECs were used between passages 4-7. HAECs were plated on glass cover slips and incubated overnight with or without added HN. After this, the cells were incubated with 10 μM dihydroethidine (DHE, Invitrogen) in Hanks' balanced salt solution buffer containing calcium, magnesium, and glucose for 30 min. at 37° C. The cells were then exposed to Ox-LDL (100 μg/ml; Biomedical Technologies Inc.) for 30 min. at 37° C. Fluorescent images were then taken with an inverted fluorescent microscope (Olympus IX70) at 15× magnification using appropriate filters fro ethidium fluorescence. Ten to twelve non-overlapping images in each culture dish were acquired and quantified by image analysis using Metamorph software (Molecular Devices). Only those cells whose fluorescence exceeded twice the basal level of fluorescence of non-treated cells were used for quantification of DHE fluorescence. Values are expressed as average relative fluorescence units for n≧100 cells per experimental condition in 2 independent experiments. Results FIG. 1.

Pretreatment with HN prior to Ox-LDL exposure resulted in a significant decrease in ROS production of approximately 50%, whereas an irrelevant peptide did not show a significant reduction in ROS formation. In addition the decrease in Ox-LDL induced ROS formation by HN occurred in a dose-dependent manner.

### Example 2

### Humanin Attenuates Ox-LDL Apoptosis in HAEC

During atherosclerosis the ongoing formation of ROS results in the induction of apoptosis in the vascular wall. Whether HN could prevent HAECs from undergoing apoptosis after Ox-LDL exposure was investigated.

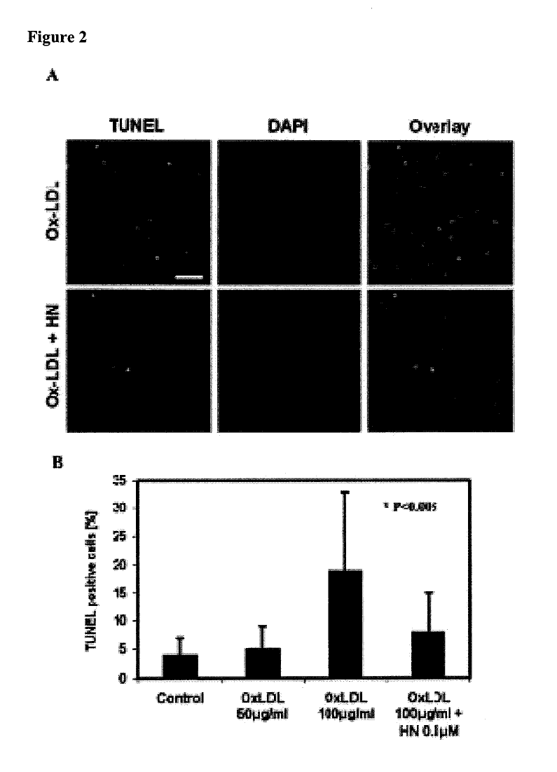

To accomplish this, a TUNEL assay was applied to measure the levels of apoptosis in HAECs treated with or without HN prior to exposure with Ox-LDL (FIG. 2). HAECs were plated on glass cover slips and incubated overnight with or without added HN (0.1 μM). The cells were then exposed to Ox-LDL (100 μg/ml; Biomedical Technologies Inc.) for 6 hours at 37° C. in the absence of HN. Apoptotic cells were quantified by TUNEL assay using the In Situ Death Detection Kit® (Roche Applied Science), mounted in SlowFade containing DAPI (Invitrogen) and observed by flourescent microscope. Apoptotic cells were defined based in morphological changes in the nuclei and the fluorescent intensity after TUNEL staining. The apoptotic index (AI)=(number of apoptotic cells)/(total number of cells)

By TUNEL assay, the basal rate of apoptosis in untreated HAECs cultures was found to be approximately 1-5%. HAECs incubated with Ox-LDL alone for 6 hours showed a concentration dependent increase in apoptosis from 5%±4% to 19.4%±14% after treatment with 50 μg/ml or 100 μg/ml Ox-LDL respectively. In contrast, HAEC that were pre-incubated overnight with 0.1 μM HN and subsequently exposed to 100 μg/ml Ox-LDL showed a relative decrease in apoptosis of more than 50% compared to cells without this pretreatment.

### Example 3

### Humanin Decreases Cellular Ceramide Levels

Ox-LDL is thought to increase the levels of cellular ceramide. C-16-ceramide is known to be involved in apoptosis. Therefore, whether HN would influence ceramide levels during Ox-LDL triggered oxidative stress was tested.

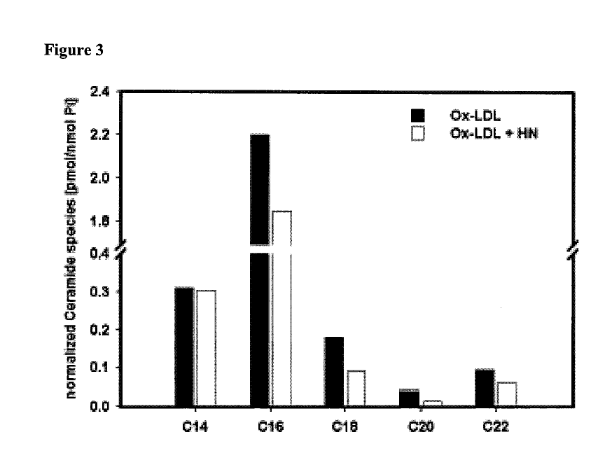

HAECs were plated on glass cover slips and incubated overnight with or without added HN (0.1 μM). The cells were then exposed to Ox-LDL (100 μg/ml; Biomedical Technologies Inc.) for 6 hours. Cells were then scraped into PBS, pelleted and frozen in liquid nitrogen. These samples were sent to MUSC-lipidomics core (Lipidomics Analytical Unit) for processing to detect and quantify individual ceramide species by HPLC/MS/MS. Pretreatment with HN resulted in a decrease in total cellular amount of ceramides, including C16-ceramide (FIG. 3).

### Example 4

### The Effect of the HN Analogue HNGF6A on Vascular Function

The effect of HNGF6A on mice was studied in 4 different animal groups (n=12 per group); Group 1: C57BL/6 feed normal diet+intraperitoneal (IP) saline, Group 2: C57BL/6 feed normal diet+IP HNGF6A, Group 3: ApoE-deficient feed high cholesterol diet+IP saline, and Group 4: ApoE-deficient feed high cholesterol diet+IP HNGF6A (Group 4). Experiment was conducted over the course of 16 weeks.

Female C57BL/6 mice and Apo E-deficient mice were obtained at the age of 4 weeks from Jackson Laboratory Animals had free access to water, were maintained at 24° C., and kept at a 12 hr light/dark cycle. Depending on the study group, the mice were treated with a normal diet or high cholesterol diet (HC, 0.15% cholesterol and 42% milk fat by weight, TD88137 or “Western diet”; Harlan Teklad) and received intraperitoneal injection (IP) of saline or HNGF6A (glycine variant of HN, 0.4 mg/kg/day, Peptide International, Louisville, Ky.) for 16 weeks. HNGF6A is 1000 times more potent than normal HN. Blood samples were collected by cardiac puncture.

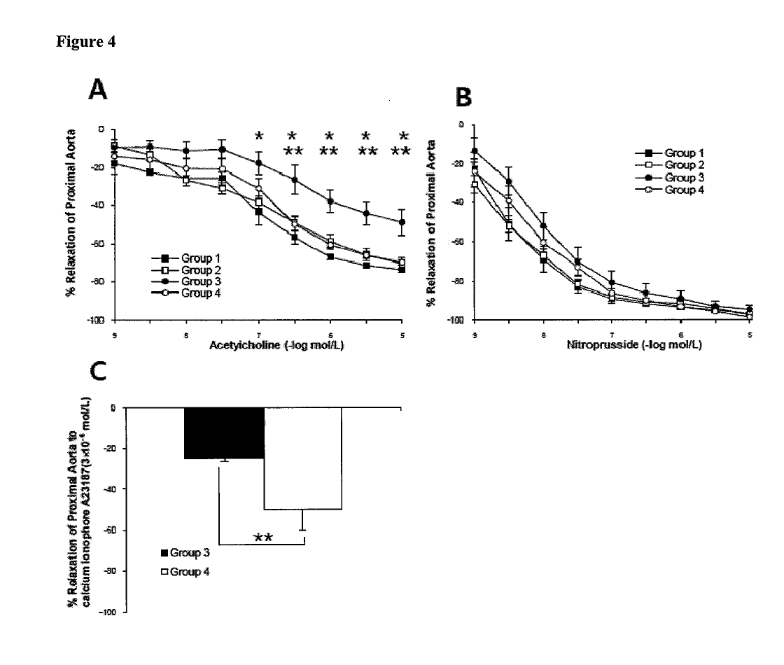

Following this, the entire thoracic aorta was placed into ice cold (4° C.) Krebs Ringer bicarbonate solution (in mmol/L: NaCl 118.6, KCl 4.7, CaCl₂ 2.5, KH₂PO₄ 1.2, NaHCO₃ 25.1, edentate calcium disodium 0.026, glucose 11.1) and rinsed with a cannula to remove residual blood. Perivascular tissue was removed carefully to avoid damage to the endothelial surface and vessels were cut into rings (3 to 4 mm long). The first aortic ring from just distal to left subclavian artery was defined as proximal aorta. Aortic arches were embedded in paraffin for histologic examination. Proximal aortas were also embedded in paraffin after organ chamber experiment. Rings were suspended in organ chambers containing 10 mL of Krebs solution (37° C., pH 7.3) and aerated with a mixture of 94% O₂ and 6% CO₂. Isometric tension was continuously recorded. The resting tension was gradually increased to reach the final tension of 1.6 g. After equilibration for 1 h at a resting tension, all vessels were examined for viability by contractile responses to 20 mmol/L KCl twice. Each time after contraction, the KCl was washed out and incubated for an additional 30 minutes. Aortic rings were precontracted with phenylephrine (Sigma-Aldrich). After stabilization of submaximal contraction (approximately 60˜70% of maximum) to phenylephrine, relaxation to acetylcholine (10⁻⁹ to 10⁻⁵, mol/L, Sigma-Aldrich), calcium ionophore A23187 (10⁻¹⁰ to 3×10⁻⁶ mol/L, Enzo Life Sciences, Plymouth Meeting, Pa.), and sodium nitroprusside (10⁻⁹ to 10⁻⁵ mol/L, Sigma-Aldrich) were recorded. Complete relaxation of each ring was obtained by exposure of the tissue to 10⁻⁴ mol/L papaverine (Sigma-Aldrich). Relaxations were expressed as a percentage of maximal relaxation induced by papaverine. FIG. 4.

To find out whether HNGF6A had direct vasoactive effects, excess internal mammary artery (IMA) segments were collected from patients undergoing coronary artery bypass surgery and were stored in oxygenated Krebs Ringer solution. As previously described, 4 mm rings of tissue were dissected and transferred to organ chambers with 25 mL of Krebs solution. After human IMAs were contracted with 10⁶ mmol/L phenylephrine and equilibration, arteries were relaxed with 10⁵ mol/L HNGF6A or saline as control.

### Example 5

### The Effect of the HN Analogue HNGF6A on the Histology of Mice in Groups 1-4

Morphometric analyses on hematoxylin and eosin-stained slides were performed for the measurement of plaque size using a digital image system (Nikon DXM 1200). Plaque sizes were analyzed in 4 different cross sections of each proximal aorta with MetaImaging series 6.1; Metamorph (Universal Imaging Corporation, Downingtown, Pa.).

Proximal aorta cross sections showed the presence of atherosclerotic plaques in both Group 3 and 4. Plaque size in tissue samples from Group 3 animals was significantly larger (0.09±0.01 mm²) as compared to tissue from Group 4 (0.01±0.003 mm²) Plaques were not visible in tissue samples from animals in Groups 1 and 2.

### Example 6

### The Effect of the HN Analogue HNGF6A on the Aortic Arch Apoptosis

Terminal deoxynucleotidyl transferase dUTP nick end labeling (TUNEL) was performed using the in situ Apoptosis Detection Kit (S7100-KIT; Chemicon). The procedure was performed following the manufacturers' protocol. To briefly explain the process, paraffin-embedded sections were dewaxed and rehydrated. Proteinase K (10 μg/mL in 0.1 M Tris, 50 mM EDTA, pH 8) was applied to the sections and incubated for 15 minutes at room temperature. After washing, two drops of equilibration buffer was applied to the sections for 0.5 to 1 minutes to facilitate penetration of terminal dUTP transferase, the TdT enzyme. A reaction mixture was prepared by mixing the reaction buffer and TdT enzyme at 5:1. Then this reaction mixture was applied to the sections, which were incubated in a humidified chamber for 1 hour at 37° C. After the incubation, the sections were washed in stop buffer 1 mL stock in the kit+34 mL distilled water). Two drops of peroxidase-conjugated anti-DIG antibody was applied to the sections for 30 minutes at room temperature followed by incubation with Diaminobenzidine+substrate-chromogen (DakoCytomation) for 3 minutes. The sections were counterstained with methyl green followed by dehydration and mounting.

The number of positive cells in atherosclerotic plaque was counted using x400 magnification. Mice from Group 3 showed a significantly larger number of apoptotic cells (197±41 cells/mm²) as compared to mice in Group 4 (84±17 cells/mm²) in plaques in the aortic arch (P<0.05).

Results are presented as the number of cells divided by plaque area. Data was expressed as mean±SEM. Comparison of different groups was performed by one-way ANOVA followed by post-hoc tests for parametric and nonparametric distribution. Comparison between the two groups was made by student's T-test or Mann-Whitney rank sum test. A value of P<0.05 was considered significant.

### Example 7

### The Effect of the HN Analogue HNGF6A on Plasma Lipids and Cytokines

Blood samples from each group of animals were immediately transferred to EDTA tubes, centrifuged at 5,000 rpm for 10 min, and kept at −80° C. until determination. Total cholesterol (T.Chol), high density lipoprotein-cholesterol (HDL-Chol), and triglyceride (TG) were measured using Cobas c311 analyzer (Roche Diagnotics, Indianapolis, Ind.). Plasma interleukin (IL)-6, monocytes chemotactic protein (MCP)-1, tumor necrosis factor (TNF)-α, vascular endothelial growth factor (VEGF), insulin, leptin, resistin, and tissue plasminogen activator inhibitor (tPAI)-1 were measured by the Inflammation Core Laboratories of the Diabetes and Endocrinology Research Center at the University of California, Los Angeles: by the LINCOplex assay for Mouse Cytokines and Adipokines.

The plasma level of t-PA and VEGF were found to be significantly higher in Group 3 and Group 4 relative to Group 1, but unaffected by HNGF6A treatment.

### Example 8

### Plasma HN Level According to Coronary Endothelial Function in Humans

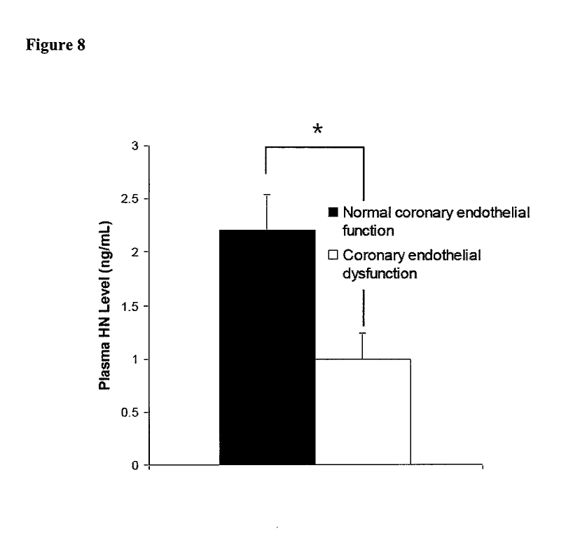

Plasma HN levels were measured in 20 patients with normal coronary endothelial function and 20 patients with coronary endothelial dysfunction. Age, sex, smoking diabetes mellitus, hypertension, body mass index, total cholesterol, LDL-cholesterol, HDL-cholesterol and TG level were not different between the two groups. Plasma HN level was significantly lower in patients with coronary endothelial dysfunction (1.28±0.25 ng/mL) as compared to normal coronary endothelial function group (2.20±0.33 ng/mL); P<0.05. FIG. 8.

We claim:

1. A method for treating or reducing the progression of atherosclerosis in a subject, the method comprising administering to a subject a composition comprising humanin (HN) or a HN analogue in an amount effective to reduce the progression of atherosclerosis as compared to a subject untreated with HN or HN analogue.

2. The method of claim 1, wherein the HN analogue is selected from the group consisting of SEQ ID NOs: 2-19.

3. The method of claim 1, wherein the HN analogue is SEQ ID NO: 16.

4. A method for reducing atherosclerotic plaque size in a subject, the method comprising administering to a subject a composition comprising humanin or a humanin analogue in an amount effective to result in reduced atherosclerotic plaque size as compared to a subject untreated with HN or HN analogue.

5. The method of claim 4, wherein the humanin analogue is selected from the group consisting of SEQ ID NOs:2-19.

6. The method of claim 4, wherein the humanin analogue is SEQ ID NO: 16.

7. A method for improving the survival of endothelial cells in vitro, the method comprising contacting the endothelial cells with a composition comprising humanin or a humanin analogue in an amount effective to improve survival of the endothelial cells as compared to an untreated population of endothelial cells.

8. The method of claim 7, wherein the humanin analogue is selected from the group consisting of SEQ ID NOs: 2-19.

9. The method of claim 4, wherein the humanin analogue is SEQ ID NO: 16.

10. The method of claim 1, wherein the humanin or humanin analogue is administered by administering a vector encoding the humanin or humanin analogue to the individual such that the humanin or humanin analogue is expressed from the vector.

11. A method for measuring endothelial function in a subject, the method comprising measuring the humanin concentration from the bodily fluid of a subject, comparing the concentration of HN in the bodily fluid from the subject with that of a control subject or value and based in the results of the comparison starting a treatment regimen.

12. The method of claim 11, wherein the treatment regimen is the administration of an effective amount of HN or HN analogue.

13. The method of claim 11, wherein the bodily fluid is plasma.

14. A method for reducing endothelial dysfunction in a subject, the method comprising administering to a subject a composition comprising humanin (HN) or a HN analogue in an amount effective to reduce endothelial dysfunction as compared to a subject untreated with HN or HN analogue.
